(19) United States
(12) Patent Application Publication
(10) Pub. No.: US 2008/0015675 A1
(43) Pub. Date: Jan. 17, 2008

(54) Balloon catheter system and method

(75) Inventors: Scott William Courts, Pleasanton, CA (US); Peter S. Brown, Palo Alto, CA (US)

Correspondence Address: Intellectual Property Department;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/485,128

(22) Filed: Jul. 11, 2006

(51) Int. Cl. A61F 2/06

(52) U.S. Cl. 623/1.11

(57) ABSTRACT

A balloon catheter system includes a balloon dilation catheter including a balloon at a distal part thereof. The catheter is defined at least in part by at least part of a tubular element. The catheter has a proximal part of larger diameter than a reduced diameter part of a distal part of the catheter. The system also includes a sheath, the catheter being disposed in and having a limited range of longitudinal movement relative to the sheath. A method of catheterization is also disclosed.

### BACKGROUND AND SUMMARY

The present invention relates generally to balloon catheter systems and methods and, more particularly, to balloon catheter systems and methods including sheaths.

Stent implantation procedures, particularly those for drug-eluting stents, are typically performed with predilation of a blood vessel using a balloon catheter. A guidewire is introduced into the patient's vasculature and an angioplasty balloon in a deflated condition is moved up the guidewire to the site to be dilated. When the balloon is properly positioned, the balloon is inflated to dilate the blood vessel. When the blood vessel is dilated, the balloon is deflated and withdrawn along the guidewire. A stent delivery balloon catheter is then inserted in the dilated vessel over the guidewire, the stent is expanded within the vessel, and the balloon and the guidewire are withdrawn.

A disadvantage of known stent implantation procedures is that they tend to be time consuming and involve a number of separate steps. Known over the wire and rapid exchange catheters for delivery of stents have relatively large crossing profiles or delivery diameters and it can be difficult to pass them through constricted vessels or lesion sites. Where reference is made herein to diameters of tubular members, it will be appreciated that the members may not actually be circular, particularly when compressed or otherwise under load. Frequently but not always the expression “delivery diameter” will be used herein to express a dimension of a tubular component that might not always be circular in cross-section to provide some sense of the dimensions of the component.

Another stenting procedure called direct stenting involves implantation of a stent without predilation by an angioplasty balloon. Direct stenting requires that the balloon and stent together pass through the constricted portion of the blood vessel or lesion site. When the balloon and the stent arrive at the desired location, the balloon is inflated to expand the stent. The balloon is then deflated and withdrawn, leaving the stent behind. While the direct stenting procedure can involve fewer steps and take less time than traditional stenting following angioplasty, direct stenting is generally not recommended for use with coated drug-eluting stents because the coatings can be damaged when passed through small openings.

It is desirable to provide a stent delivery system and method that can be used to implant a stent quickly and in a minimal number of steps. It is also desirable to provide a stent delivery system and method that requires a minimal crossing profile or delivery diameter such that the system and method can be used to introduce a stent to a desired location in spite of constricted blood vessel openings or tight lesions. It is also desirable to provide a balloon catheter system that facilitates replacing a first balloon catheter system with a second catheter system.

According to an aspect of the present invention, a balloon catheter system comprises a balloon dilation catheter comprising a catheter shaft and an inflatable balloon at a distal end of the catheter shaft, a sheath having a proximal end that is removably attachable to a distal end of the adapter, the sheath being adapted to be disposed around at least part of the catheter, and a guidewire disposed in and having a limited range of longitudinal movement relative to the balloon.

According to another aspect of the present invention, a balloon catheter system, comprises a balloon dilation catheter comprising a balloon at a distal part thereof, the catheter being defined at least in part by at least part of a tubular element, the catheter having a proximal part of larger diameter than a reduced diameter part of a distal part of the catheter, and a sheath, the catheter being disposed in and having a limited range of longitudinal movement relative to the sheath, the catheter being freely movable relative to the sheath rearwardly of a forwardmost position beyond which the catheter cannot move.

According to still another aspect of the present invention, a catheterization method comprises providing a balloon catheter system, comprising a balloon dilation catheter comprising a catheter shaft, and a balloon at a distal end of the catheter shaft, a guidewire disposed in and having a limited range of longitudinal movement relative to the inner tubular element, and a sheath adapted to be disposed around at least part of the catheter. The balloon, guidewire, and sheath are passed together through the patient's vasculature until the balloon is at a first location in a blood vessel. At the first location forward of a distal end of the sheath, the balloon is expanded. The sheath is advanced over the balloon at the first location. After advancing the sheath over the balloon, the catheter is withdrawn from the sheath while leaving the sheath in place.

### BRIEF DESCRIPTION OF THE DRAWINGS

The features and advantages of the present invention are well understood by reading the following detailed description in conjunction with the drawings in which like numerals indicate similar elements and in which:

FIG. 1 is a cross-sectional view of a portion of an advanceable, non-removable guide wire balloon catheter delivery system for a stent, showing a balloon in a deflated condition, according to an embodiment of the present invention;

FIG. 2 is a cross-sectional view of a portion of an advanceable, non-removable guide wire balloon catheter delivery system for a stent, showing a balloon in an inflated condition, according to an embodiment of the present invention;

FIG. 3 is a cross-sectional view of a portion of an advanceable, non-removable guide wire balloon catheter delivery system for a stent including an external insertion stop;

FIG. 4 is a cross-sectional view of a portion of an advanceable, non-removable guide wire balloon catheter delivery system for a stent according to an embodiment of the present invention;

FIG. 5 is a cross-sectional view of a portion of an advanceable, non-removable guide wire balloon catheter delivery system for a stent according to another embodiment of the present invention;

FIG. 6 is a cross-sectional view of a portion of a balloon catheter system including a sheath according to an embodiment of the present invention;

FIG. 7 is a cross-sectional view of a lock for a balloon catheter system including a sheath according to an embodiment of the present invention; and

FIGS. 8A-8C show steps in a method for catheterization according to an embodiment of the present invention.

### DETAILED DESCRIPTION

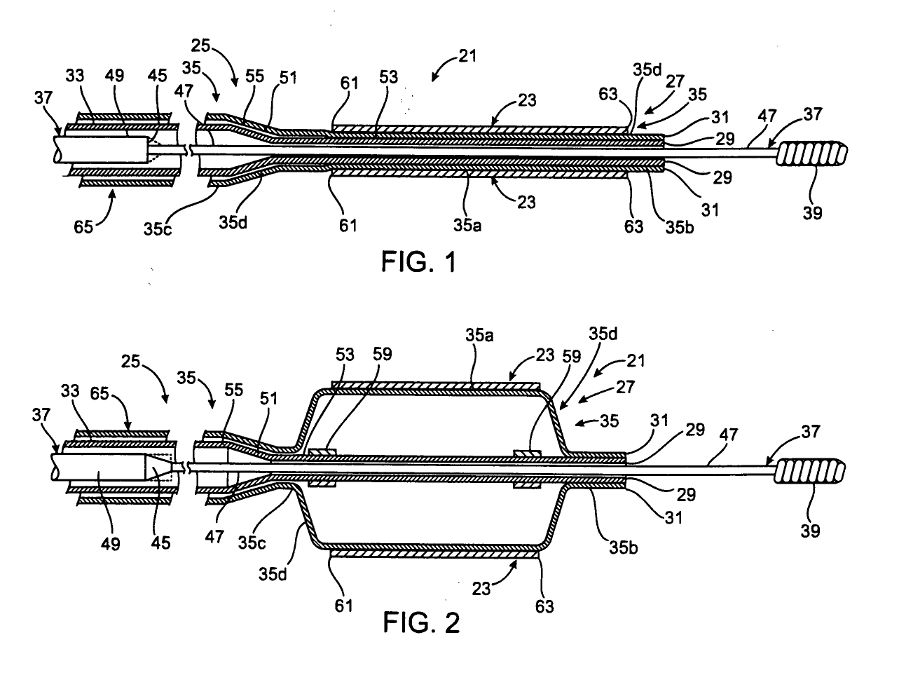

An advanceable, non-removable guide wire balloon catheter delivery system 21 for a stent 23 is shown in FIGS. 1 and 2. The system 21 comprises a balloon dilation catheter 25 comprising a balloon 27 defined by at least parts of an inner tubular element 33 and an outer tubular element 35, respectively. Distal parts 29 and 31 of the inner tubular element 33 and the outer tubular element 35 are sealed together and form part of a distal tip of the catheter 25. The outer tubular element 35 of the catheter typically includes an expandable balloon portion 35a, a distal balloon leg 35b, a proximal balloon leg 35c, and shoulder portions 35d between the expandable balloon portion and the legs. The proximal balloon leg 35c is ordinarily connected to a shaft 65 of the catheter 25, and the catheter shaft 65 is ordinarily in the form of another outer tubular member disposed outside of the inner tubular member 33 and proximal of the outer tubular member 35 forming part of the balloon 27. The catheter shaft 65 can be any suitable material, such as plastic, metal, combinations of plastic and metal, and may comprise any suitable structure, such as coils, braids, and the like. FIG. 1 shows the balloon 27 in a deflated condition while FIG. 2 shows the balloon in an inflated condition. U.S. Pat. No. 4,616,653, which is incorporated by reference, discloses a type of balloon dilation catheter with non-removable guide wire of a type generally suitable for use in connection with the balloon catheter delivery system 21. In an aspect of the invention, the delivery system 21 and stent 23 can have a small or low crossing profile or delivery diameter relative to traditional over the wire and rapid exchange catheters for delivery of stents.

The system 21 also includes a guidewire 37 disposed in and having a limited range of longitudinal movement relative to the inner tubular element 33. The guidewire 37 can be rotated relative to the inner tubular element 33. The longitudinal movability and rotatability of the guidewire 37 relative to the inner tubular element 33 facilitates accessing tight lesions and maneuvering curves or branches in a patient's vasculature. In the system 21 according to present invention, the guidewire 37 is longitudinally movable relative to the inner tubular element 33, ordinarily up to about 15 cm, although the guidewire may be movable relative to the inner tubular element over a greater or lesser distance. Typically, however, the guidewire 37 will be longitudinally movable relative to the inner tubular element 33 between about 3 cm and about 10 cm. The guidewire 37 is movable relative to the catheter 25, but is not removable. A tip 39 is typically secured to a distal part of the guidewire 37, the tip having a larger diameter than the guidewire, and is too large to be drawn through the distal end of the inner tubular element 33, thus preventing removal of the guidewire from the catheter 25. The tip 39 can be in any suitable form, such as in the form of a coil to which a semi-spherical tip is secured as disclosed in U.S. Pat. No. 4,616,653.

An expandable stent 23 is mounted on the balloon 27. The stent 23 may be a drug-eluting stent. A non-coated, drug eluting stent of the type described in WO 03/015664, which is incorporated by reference, which has drug inlays in reservoirs within the stent, is anticipated to be particularly well-suited for use in connection with the present invention because there is no coating which can be damaged or scraped off while passing the stent through small openings such as tight lesions. Other types of uncoated stents that are likely to be well-suited for use in connection with the present invention include bioresorbable drug impregnated stents and stents in which drugs are provided in channels or grooves in the stents.

It is possible to construct the system 21 in a manner that minimizes the crossing profile or delivery diameter of the stent 23 mounted on the balloon 27. For example, a distal part 47 of the guidewire 37 can be provided with at least a portion having a narrower diameter than a proximal part 49. The inner tubular element 33 and the outer tubular element 35 around the narrowed portion of the distal part 47 of the guidewire 37 can have a reduced diameter relative to portions of the inner tubular element and the outer tubular element (or catheter shaft 65 attached to outer tubular element) around larger diameter portions of the guidewire 37. As a consequence, the expandable stent 23 can have a reduced crossing profile or delivery diameter relative to systems such as those wherein a stent is implanted via a balloon catheter that is moved along a constant diameter guidewire such as over-the-wire catheterization or rapid catheter exchange. This can facilitate use of a stent in circumstances where blood vessels or lesions are too constricted to permit a stent to be implanted by conventional techniques without predilation.

Largely because of the ability of the advanceable, non-removable guide wire balloon catheter delivery system 21 for a stent 23 to have a minimal crossing profile or delivery diameter, it can be of particular use in connection with direct stenting procedures in which a stent is implanted in a patient without predilation with an angioplasty balloon. In a direct stenting procedure, the balloon 27 and stent 23 together pass through a constricted portion of a blood vessel or lesion site. When the stent 23 is positioned as desired, the balloon 27 is inflated, expanding the stent. The balloon 27 can then be deflated and the catheter system 21 can be withdrawn together with the guidewire 37, leaving the expanded stent 23 in place.

Thus, a direct stenting procedure with an advanceable, non-removable guide wire balloon catheter delivery system 21 for a stent 23 can be faster, or at least involve fewer steps, than a stenting procedure that requires, e.g., introducing, over a guidewire, a balloon catheter to the blood vessel for predilation of the blood vessel followed by removal of the balloon catheter and introduction of a stent over the same guidewire. A non-coated, drug eluting stent 23 of the type having the drug within the stent is of particular use in connection with a direct stenting procedure using the advanceable, non-removable guide wire balloon catheter delivery system 21 for a stent because a coating on the stent is not apt to be damaged or scraped off while passing the stent through small openings such as tight lesions.

In direct stenting operations, it is generally desirable to use balloons with high rated burst pressures (RBP). Typical RBPs for known balloons is about 16-18 atmospheres. The higher a balloon's rated burst pressure, the more it is likely to be able to fully expand a stent in an undilated lesion. This is important, of course, because, if the stent does not fully expand, it may be difficult to remove the balloon and it may have to be surgically removed. Also, even if the balloon can be removed when a stent has not been fully expanded, it will still be necessary to get another balloon into the stent to expand it, which can be very difficult.

Typical RBPs for known balloons is about 16-18 atmospheres. According to aspects of the present invention, the RBP of a balloon can be higher than typical RBPs while having the same or a reduced crossing profile or diameter as the known balloons at least because, according to aspects of the present invention, the reduced diameter portion 53 of the inner tubular element 31 and the reduced diameter portion 47 of the guidewire 37 permit use of an outer tubular element 35 having thicker walls than is typical, while still maintaining a small crossing profile or delivery diameter. Thicker walls in the outer tubular element can facilitate operation of balloon catheter delivery systems according to aspects of the present invention with balloons having higher RBPs, i.e., RBPs above those of typical balloon catheters, such as RBPs in the range of greater than 18 and, using presently available materials, likely up to RBPs of about 20 atmospheres or more. The higher a balloon's rated burst pressure, the more it is likely to be able to fully expand a stent in an undilated lesion. This is important, of course, because, if the stent does not fully expand, it may be difficult to remove the balloon and it may have to be surgically removed. Also, even if the balloon can be removed when a stent has not been fully expanded, it will still be necessary to get another balloon into the stent to expand it, which can be very difficult.

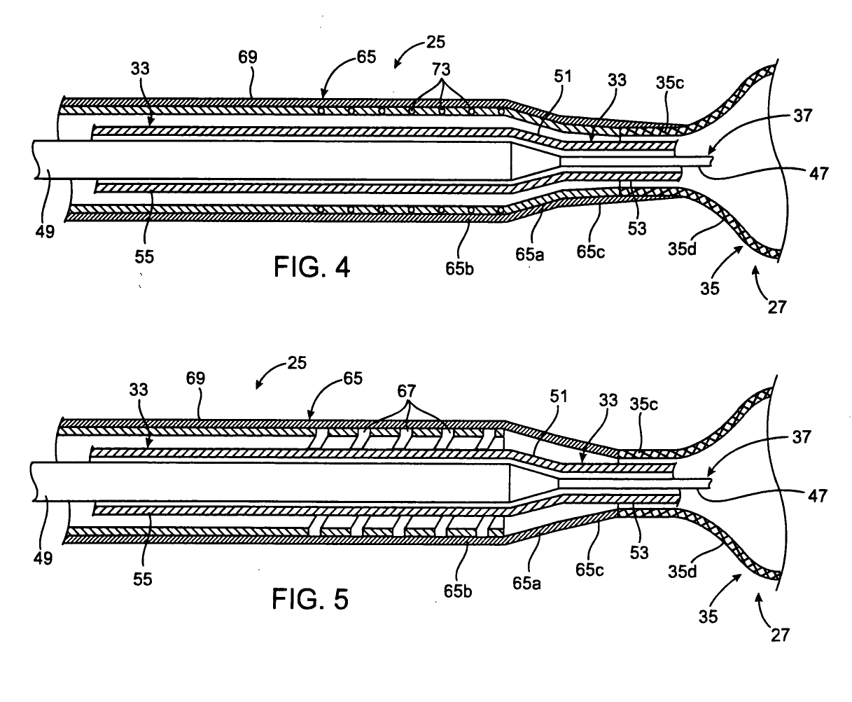

The advanceable, non-removable guide wire balloon catheter delivery system 21 for a stent 23 comprises what shall be denominated an insertion stop for limiting relative longitudinal forward movement of the guidewire 37 inside the inner tubular element 33. An embodiment of an insertion stop is seen in FIGS. 1 and 2 and comprises a guidewire transition 45 between a narrower diameter portion 47 and a larger diameter portion 49 of the guidewire 37. The illustrated embodiment of the insertion stop also comprises an inner tubular element transition 51 between a narrower diameter distal portion 53 and a larger diameter proximal portion 55 of the inner tubular element 33. As seen in FIGS. 4-5, the catheter shaft 65 may have a transition 65a from a larger diameter portion 65b to a reduced diameter portion 65c where it meets the outer tubular member 35 to facilitate keeping the crossing profile or delivery diameter of the system at a minimum.

Another form of stop that can be provided with the balloon catheter delivery system 21 prevents relative longitudinal rearward movement, i.e., retraction, of the guidewire 37 beyond a particular position relative to the balloon 27 and is herein denominated a retraction stop. In an embodiment, such a stop is provided where the proximal end of an enlarged portion of the guidewire 37, such as a proximal end of the tip 39, comes into contact with the distal ends 29 and 31 of the inner and outer tubular elements 33 and 35 or another tip of the catheter.

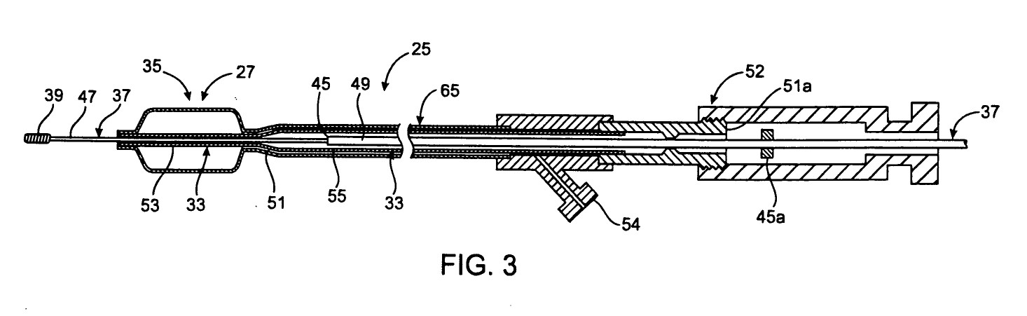

When the guidewire transition 45 contacts the inner tubular element transition 51, the guidewire 37 may be in its forwardmost position relative to the inner tubular element 33. As seen in FIG. 3, an “additional” insertion stop, hereinafter denominated as an external insertion stop, which might be in a form comprising another guidewire transition 45a and another transition 51a, can be provided in the balloon catheter delivery system 21. This “additional” or external insertion stop may be disposed in a position in which it is likely that the external insertion stop will be disposed outside of the patient's body and is therefore denominated an “external” stop, although it might also be provided so that it is likely to be disposed inside of the patient's body. A stop external to the patient's body is described here for purposes of illustration, and it will be appreciated that the references to an external stop or an external transition are not intended to limit the present invention. The additional, external transition 51a can be provided in the inner tubular element 33 or, as seen in FIG. 3, the additional, external transition 51a can be disposed proximal the inner tubular element 33, such as in an adapter 52, proximal an inflation port 54. As seen in FIGS. 1 and 2, the insertion stop can be in a position in which it will be disposed inside of the patient's body, such as near the proximal part 35c of the balloon 27. When an external insertion stop is used, it is ordinarily not an additional stop but, rather, is the only stop, and the internal reduced diameter components such as the internal guidewire transition 45 and the internal inner tubular element transition 51 forming what is denominated herein as the insertion stop will mainly serve the function of permitting a reduced diameter of the guidewire 37 and inner tube 33 under the balloon 27 so that the stent 23 crossing profile or delivery diameter dimensions can be minimized, i.e., the internal guidewire transition 45 may approach but will not ordinarily contact the inner internal tubular element transition 51. By providing the insertion stop by or before the proximal part 35c of the balloon 27, narrow diameter portions 47 and 53 of the guidewire 37 and the inner tubular element 33 can be disposed by the balloon 27 which will permit the crossing profile or delivery diameter of the balloon and stent to be minimized. Thus, the crossing profile or delivery diameter of the delivery system 21 and stent 23 can be small or low relative to known over the wire and rapid exchange catheters for the delivery of stents.

As seen in FIGS. 1-2, the guidewire transitions 45 and the inner tubular element transitions 51 can be abrupt (solid lines in FIG. 1, phantom in FIG. 2) or tapered (phantom in FIG. 1, solid lines in FIG. 2). However, it is presently expected that, ordinarily, guidewire transitions 45 and inner tubular element transition 51 will be gradual, tapered transitions providing gradual changes in the catheter's flexibility, and external transition 51a will be abrupt. The more abrupt the transition from one diameter to another, the less surface area is available to result in friction between outside surface of the guidewire transition 45a and the inside surface of the transition 51a. An abrupt transition can facilitate rotating the guidewire 37 relative to the inner tubular element 33. If an exterior insertion stop is provided, the interior transition portions will typically not be able to contact, and they may be gradual or tapered, while the exterior insertion stop will typically be abrupt to facilitate rotation of the guidewire 37 relative to the inner tubular element 33. Of course, any combination of tapered or non-tapered transitions can be used, as well. If a taper is provided to transition from a larger to a smaller diameter portion 55 and 53 of the inner tubular element transition, the taper will ordinarily extend over a length of about 0.1 cm to about 3 cm.

As seen in FIG. 2, the advanceable, non-removable guide wire balloon catheter delivery system 21 can be provided with at least one radiopaque marker 59 disposed on the inner tubular element 33 proximate at least one end 61 and/or 63 of the stent, such as to facilitate determining a location of the stent 23 inside a patient. A marker 59 can be provided at any desired location. For example, a plurality of markers 59 can be provided, such as at opposite ends of the stent 23 to facilitate precisely determining the location of the ends 61 and 63 of the stent. The marker 59 does not, however, have to be placed at either end of the stent 23.

It is desirable that the advanceable, non-removable guide wire balloon catheter delivery system 21 be capable of being pushed through small openings. U.S. patent application Ser. No. 11/251,236, filed Oct. 13, 2005, entitled RAPID EXCHANGE CATHETER WITH HYPOTUBE AND SHORT EXCHANGE LENGTH is incorporated by reference and discloses catheter systems using laser-cut hypotubes due to their excellent pushability and small diameter. The advanceable, non-removable guide wire balloon catheter delivery system 21 can include a catheter shaft 65 as seen in FIG. 5 that can comprise at least one of hypotubes or laser cut hypotubes 67 at least partially covered with a polymer sleeve which is bonded to the proximal balloon leg 35c. Instead of or in addition to hypotubes or laser cut hypotubes 67 and wall thickness variations, the catheter shaft 65 can include braids or coils 73, as seen in FIG. 4. The braids and coils may be incorporated in a polymer shaft which may optionally have a wall thickness at a proximal part 69 of the catheter shaft greater than a wall thickness of a distal part 71 of the catheter shaft.

In a method according to an aspect of the present invention, a direct stenting method of implanting a stent 23 in a patient is provided. According to the method, an advanceable, non-removable guide wire balloon catheter delivery system 21 for a stent 23 is provided, the system comprising a balloon dilation catheter 25 comprising a balloon 27 defined by at least parts of distal parts 29 and 31 of an inner tubular element 33 and an outer tubular element 35. The system 21 further comprises a guidewire 37 disposed in and having a limited range of longitudinal movement relative to the inner tubular element 33 and a full range of rotational movement. The system 21 further comprises an expandable stent 23 mounted on the balloon 27. The stent 23 mounted on the balloon 27 in a deflated condition (FIG. 1) is passed through the patient's vasculature to a desired location. The stent 23 is then expanded at the location by inflating the balloon 27.

Without intending to limit the present invention to components having any specific dimensions, following are typical dimensions for components for use in connection with embodiments of the present invention. A stent 23 for use in connection with the present invention is typically 8-40 mm (0.31-1.57 inches) in length and has an unexpanded diameter of 1.01 mm (0.040 inches) or less, and preferably 0.094 mm (0.037 inches) or less, when fully crimped. The stent 23 typically has an expanded diameter of 2-5 mm (0.08-0.20 inches) when fully expanded by the balloon. The balloon portion 35a of the outer tubular member 35 is typically about 1-2 mm (0.04-0.08 inches) longer than the stent 23 and has a diameter of 0.63-1.0 mm (0.025-0.040 inches) when uninflated and a diameter sufficient to fully deploy the stent when inflated. The balloon portion 35a typically has a wall thickness of approximately 0.01-0.03 mm (0.0005-0.0013 inches) when deflated, although the balloon will often be folded, such as in a tri-fold, quad-fold, or more folds, for delivery, which can add to delivery diameter.

The proximal balloon leg 35c typically has an outside diameter of 0.64-0.90 mm (0.025-0.035 inches) and a wall thickness of about 0.06-0.25 mm (0.002-0.010 inches). The distal part of the catheter shaft 65 proximate the proximal balloon leg 35c can have an outside diameter of 0.066-1.02 mm (0.026-0.40 inches) where it transitions to the proximal balloon leg. This distal part of the catheter shaft 65 may step down in diameter as shown in FIGS. 4 and 5 to follow the step down in diameter to the narrower diameter portion 53 of the inner tubular element 33.

The narrow diameter portion 53 of the inner tubular element 33 can have an outside diameter of 0.03-0.58 mm (0.012-0.023 inches and a wall thickness of 0.05-0.13 mm (0.002-0.005 inches), and the larger diameter portion 55 of the inner tubular element can have an outer diameter of 0.51-0.76 mm (0.020-0.030 inches) (or about 1.5 to 2 times the diameter of the narrow diameter portion) and a wall thickness of 0.05-0.18 mm (0.002-0.007 inches). A length of the transition portion 51 of the inner tubular element 33 is typically between 0.01-3 cm (0.004-1.18 inches). The transition portion 51 of the inner tubular element 33 is typically located 1-5 cm (0.4-2 inches) from the proximal balloon leg 35c. The transition portion 51 of the inner tubular element 33 can be proximal of or at any transition 65a in the catheter shaft 65 hypotube 67. The transition portion 51 of the inner tubular element 33 is typically located within 0-5 cm (0-2 inches) of the proximal balloon leg 35c.

The transition from a large diameter portion 49 of the guidewire 37 to the narrower diameter portion 47 of the guidewire will ordinarily depend upon an amount of travel desired, although it is anticipated that it will ordinarily be between 2-15 cm (0.79-5.90 inches) behind the distal part of the guidewire, i.e., behind the tip 39. The large diameter portion 49 of the guidewire 37 typically has a diameter of about 0.35-0.45 mm (0.014-0.018 inches) and the narrow diameter portion 47 typically is about 0.15-0.254 mm (0.006-0.010 inches). The narrow diameter portion 47 generally has a diameter that is no greater than 70% of the diameter of the large diameter portion 49, and more typically no greater than 60% of the diameter of the larger diameter portion.

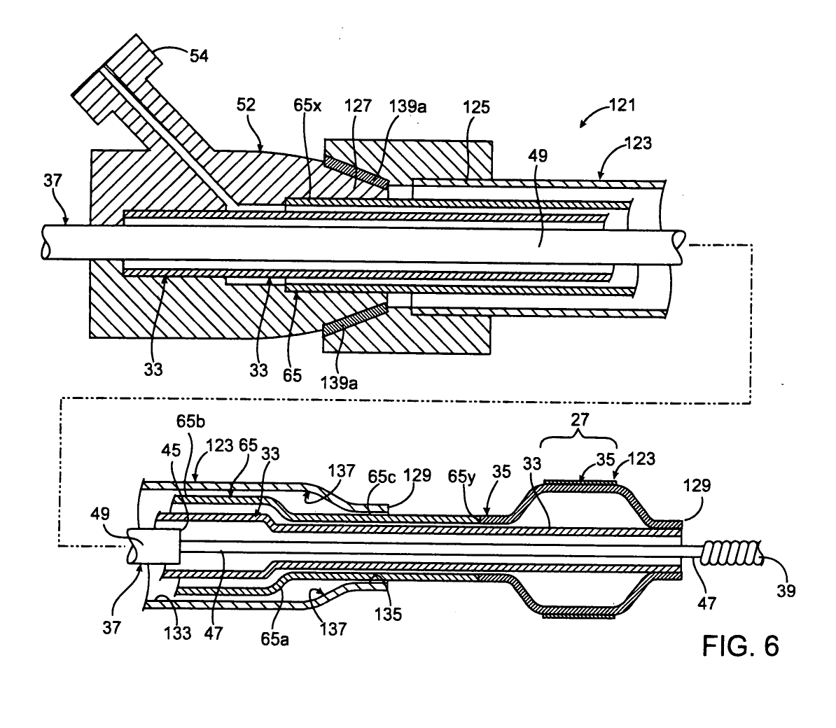

A balloon catheter system 121 that can comprise a balloon dilation catheter delivery system 21 for a stent 23 as has been described, or some other balloon dilation catheter system, is seen in FIG. 6. For purposes of illustration, the balloon catheter system 121 shall be described in conjunction with the balloon dilation catheter delivery system 21 for a stent 23, however, the balloon catheter system 121 and a related catheterization method is not necessarily limited to use in conjunction with stent delivery. The balloon catheter system 121 comprises a catheter comprising a catheter shaft 65, an adapter 52 attached to a proximal end 65x of the catheter shaft, and (as seen in FIGS. 1-5) a balloon 27 defined by at least parts of an inner tubular element 33 and an outer tubular element 35 at a distal end 65y (FIG. 6) of the catheter shaft. Ordinarily, a distal end 65y of the catheter shaft 65 will be joined to a proximal end of the outer tubular element 35 of the balloon 27 in some suitable fashion.

The balloon catheter system 121 also includes a sheath 123 having a proximal end 125 that is removably attachable to a distal end 127 of the adapter 52. The sheath 123 is adapted to be disposed around at least part of the catheter and, ordinarily, when the sheath is attached to the distal end 127 of the adapter 52, the balloon 27 will extend out of a distal end 129 of the sheath. In the illustrated embodiment, a guidewire 37 is disposed in and has a limited range of longitudinal movement relative to the inner tubular element 33 and, ordinarily, when the sheath is attached to the distal end 127 of the adapter 52, the tip 39 of the guidewire 37 will extend out of a distal end 129 of the sheath. FIG. 6 shows the balloon 27 and part of the tip 39 inside the sheath 123 while the proximal end 125 of the sheath is attached to the adapter for purposes of illustration.

The balloon catheter system 121 can also comprise a sheath taper 137 that corresponds to a taper 65a in the catheter. In the embodiment of FIG. 6, the sheath 123 comprises a large inside diameter part 133, a small inside diameter part 135, and a sheath transition 137 between the large inside diameter part and the small inside diameter part. The catheter shaft 65 comprises a first outside diameter part 65b that is larger than the small inside diameter part 135 of the sheath 123. The catheter shaft 65 ordinarily comprises a small outside diameter part 65c attached to the outer tubular element 35 and that is smaller than the first outside diameter part 65b and is adapted to be received in the small inside diameter part 135 of the sheath 123.

Longitudinal movement of the catheter relative to the sheath 123 is ordinarily limited by the tapers 65a and 137 such that the distal end of the balloon 27 is adapted to extend no further than about 20 cm (7.87 inches), preferably 10 cm (3.94 inches), past the distal end 129 of the sheath, and the distal end or tip 39 of the guidewire 37 is adapted to extend no further than about 15 cm (5.91 inches) past the distal end of the balloon, and no further than about 35 cm (13.78 inches) past the distal end of the sheath.

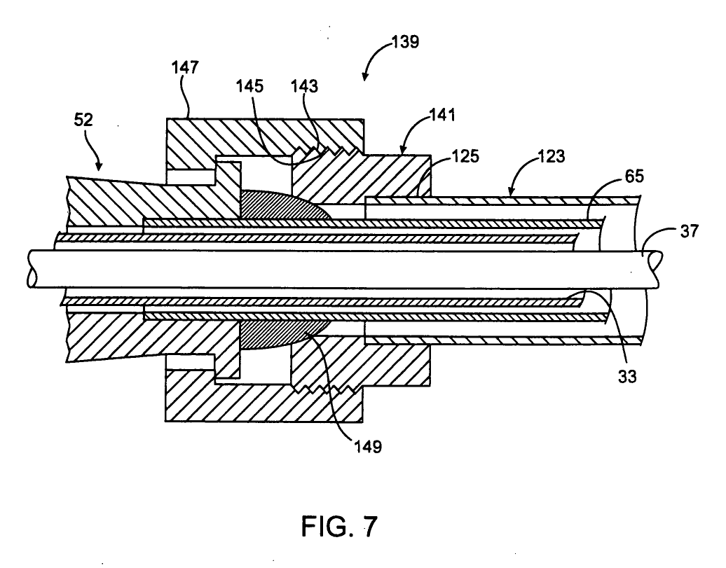

The balloon catheter system 121 can comprise a lock 139 for removably attaching the proximal end 125 of the sheath 123 to the distal end 127 of the adapter 52. The lock 139 can take any suitable form and will ordinarily be adapted to at least prevent relative axial movement of the sheath 123 and the adapter 52. The lock 139 will also ordinarily be adapted to prevent relative rotational movement of the sheath 123 and the adapter 52. While various locking arrangements are known and are suitable for use as the lock 139, as seen in FIG. 7, the lock 139 may comprise a compression tube fitting. In the illustrated lock 139, the sheath 123 and the catheter are not rotatable relative to each other, and the proximal end 125 of the sheath 123 includes a male member 141 with external threads 143 that mate with internal threads 145 of a female member 147 that may form an integral or non-integral part of the adapter 52. When the male member 141 and the female member 147 are screwed together, a compression gasket 149 is compressed around the catheter shaft 65 to secure the sheath 123 relative to the adapter 52 (and the catheter in general) so that they are not axially or rotationally movable relative to each other. While FIG. 7 shows an embodiment of a lock wherein a male member 141 is disposed at the proximal end 125 of the sheath 123 and a female member 147 is disposed at the distal end 127 of the adapter 52, it will be appreciated that other embodiments of the lock might comprise a male member disposed at the distal end 127 of the adapter and a female member at the proximal end 125 of the sheath 123, as shown schematically by the lock 139a in FIG. 6, or the lock may involve connecting structures that are not necessarily male or female. Other forms of locks suitable for use in connection with the present invention can include snap locks and detent locks.

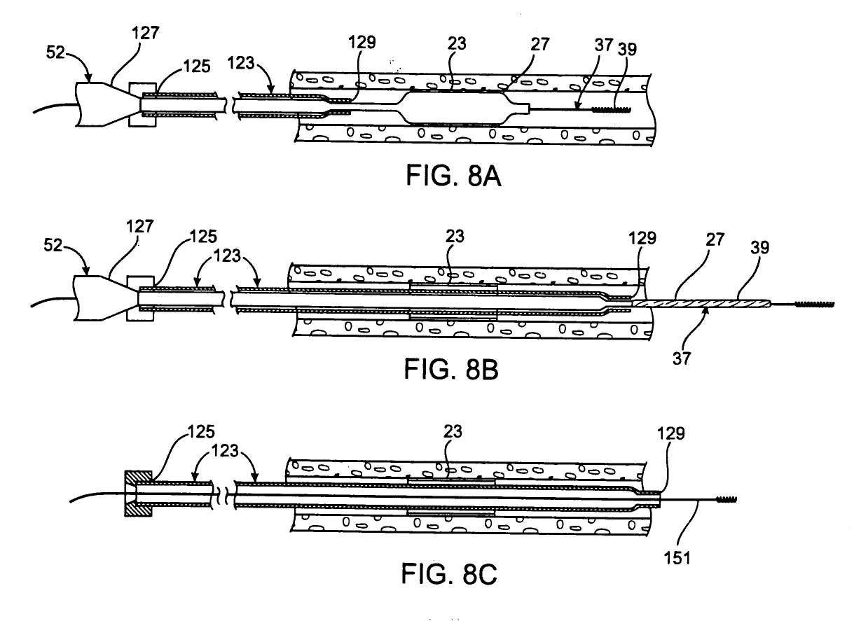

The balloon catheter system 121 facilitates performing a catheterization method as seen in FIGS. 8A-8C. As seen in FIG. 8A, the catheter can be advanced through a patient's vasculature for any purpose, such as to deliver a stent 23, such as by a direct stenting method or some other known method, such as by a self-expanding stent that expands when the sheath 123 is removed from around the stent. When the stent 23 is delivered using a fixed wire catheter or a semi-movable wire catheter, such as that described in connection with FIGS. 1-5, the balloon 27 and stent 23 are disposed forward of the distal end 129 of the sheath when the balloon is inflated and the stent is expanded.

A disadvantage of fixed wire balloon catheter or semi-movable wire balloon catheters is that, in circumstances where it is desirable to remove the balloon catheter and insert another catheter or device to the treatment site, there is no guidewire remaining behind for quick access to the treatment site. For example, when a stent is not completely deployed or a dissection occurs, it may be desirable to remove the stent delivery catheter and insert another longer or larger balloon catheter to post dilate the site. In another case, if it is determined that a stent is the improper size, it may be desirable to remove and replace the catheter and stent with a correct size before deployment of the stent. Also, if a stent is implanted and determined to be not correctly placed or not long enough, an additional procedure may be desired to add an additional stent, called an overlap. In another example, where, after a first stent placement, it is determined that another procedure is needed at a location distal of the first stent, it may be desirable to remove the first catheter and insert a second catheter and/or stent. In each of these cases, and in other similar situations, the sheath 123 of the invention provides an alternative for reaccessing a site with a second catheter in an expeditious manner. The proximal end 125 of the sheath 123 can then be detached from the distal end 127 of the adapter, and the sheath can be advanced over the balloon at the further location. Such a method might be desirable, for example, when an area of dissection in a blood vessel is disposed forward of a location to which a stent is to be delivered.

After advancing the sheath 123 over the balloon 27, the catheter can be withdrawn from the sheath while leaving the sheath in place, i.e., at or beyond the further location to which the balloon had been advanced and at which it had been expanded. As seen in FIG. 8C, after withdrawing the catheter, a second guidewire 151 can be advanced through the sheath. After advancing the second guidewire 151, the sheath 123 can be withdrawn and the second guidewire can be left in place, thereby facilitating further operations at the further location, such as advancing a second, over-the-wire type or rapid exchange type balloon catheter stent delivery system over the second guidewire. Alternatively, the second catheter can be inserted through the sheath without a second guidewire, particularly another fixed wire or semi-movable wire balloon catheter.

In the present application, the use of terms such as “including” is open-ended and is intended to have the same meaning as terms such as “comprising” and not preclude the presence of other structure, material, or acts. Similarly, though the use of terms such as “can” or “may” is intended to be open-ended and to reflect that structure, material, or acts are not necessary, the failure to use such terms is not intended to reflect that structure, material, or acts are essential. To the extent that structure, material, or acts are presently considered to be essential, they are identified as such.

While this invention has been illustrated and described in accordance with a preferred embodiment, it is recognized that variations and changes may be made therein without departing from the invention as set forth in the claims.

What is claimed is:

1. A balloon catheter system, comprising:
a balloon dilation catheter comprising a catheter shaft and an inflatable balloon at a distal end of the catheter shaft;
a sheath having a proximal end that is removably attachable to a distal end of the adapter, the sheath being adapted to be disposed around at least part of the catheter; and
a guidewire disposed in and having a limited range of longitudinal movement relative to the balloon.

2. The balloon catheter system as set forth in claim 1, comprising a sheath insertion stop for preventing the balloon from extending longitudinally beyond a predetermined distance forward of a distal end of the sheath.

3. The balloon catheter system as set forth in claim 2, wherein the sheath comprises a large inside diameter part, a small inside diameter part, and a sheath transition between the large inside diameter part and the small inside diameter part, and the catheter shaft comprises a first outside diameter part that is larger than the small inside diameter part of the sheath, the sheath insertion stop comprising the sheath transition.

4. The balloon catheter system as set forth in claim 3, wherein the catheter shaft comprises a small outside diameter part attached to the balloon and smaller than the first outside diameter part of the catheter, the small outside diameter part of the catheter being adapted to be received in the small inside diameter part of the sheath.

5. The balloon catheter system as set forth in claim 1, wherein a distal part of the guidewire has a narrower diameter than a proximal part.

6. The balloon catheter system as set forth in claim 1, wherein the guidewire is non-removable from the catheter and is longitudinally movable relative to the balloon to about 15 cm.

7. The balloon catheter system as set forth in claim 1, comprising a guidewire stop for limiting relative longitudinal movement of the guidewire and the balloon.

8. The balloon catheter system as set forth in claim 1, comprising a lock for removably attaching the proximal end of the sheath to an adapter at a proximal end of the catheter shaft.

9. The balloon catheter system as set forth in claim 8, wherein the lock prevents relative axial movement of the sheath and the adapter.

10. The balloon catheter system as set forth in claim 9, wherein the lock prevents relative rotational movement of the sheath and the adapter.

11. The balloon catheter system as set forth in claim 8, wherein the lock comprises a compression tube fitting.

12. The balloon catheter system as set forth in claim 1, comprising an expandable stent disposed around the balloon, the sheath being adapted to be disposed around the stent when the stent is in an unexpanded condition.

13. The balloon catheter system as set forth in claim 12, wherein the sheath is adapted to be disposed inside of the stent when the stent is in an expanded condition.

14. A balloon catheter system, comprising:
a balloon dilation catheter comprising a balloon at a distal part thereof, the catheter being defined at least in part by at least part of a tubular element, the catheter having a proximal part of larger diameter than a reduced diameter part of a distal part of the catheter; and
a sheath, the catheter being disposed in and having a limited range of longitudinal movement relative to the sheath, the catheter being freely movable relative to the sheath rearwardly of a forwardmost position beyond which the catheter cannot move.

15. The balloon catheter system as set forth in claim 14, comprising an expandable stent mounted on the balloon.

16. The balloon catheter system as set forth in claim 14, comprising a guidewire disposed in the tubular part and having a limited range of longitudinal movement relative to the catheter.

17. The balloon catheter system as set forth in claim 16, wherein a distal part of the guidewire disposed in the reduced diameter part of the distal part of the catheter has a reduced diameter relative to a proximal part of the guidewire.

18. The balloon catheter system as set forth in claim 17, comprising a guidewire stop for limiting relative longitudinal movement of the guidewire and the catheter.

19. The balloon catheter system as set forth in claim 14, comprising a lock for removably attaching the proximal end of the sheath to the distal end of the adapter.

20. The balloon catheter system as set forth in claim 19, wherein the lock prevents relative axial movement of the sheath and the adapter.

21. The balloon catheter system as set forth in claim 20, wherein the lock prevents relative rotational movement of the sheath and the adapter.

22. The balloon catheter system as set forth in claim 19, wherein the lock comprises a compression tube fitting.

23. The balloon catheter system as set forth in claim 14, comprising a sheath insertion stop for preventing the balloon from extending longitudinally beyond a predetermined distance forward of a distal end of the sheath.

24. The balloon catheter system as set forth in claim 23, wherein the sheath comprises a large inside diameter part, a small inside diameter part, and a sheath transition between the large inside diameter part and the small inside diameter part, and the catheter shaft comprises a first outside diameter part that is larger than the small inside diameter part of the sheath, the sheath insertion stop comprising the sheath transition.

25. The balloon catheter system as set forth in claim 24, wherein the catheter shaft comprises a small outside diameter part attached to the tubular element and smaller than the first outside diameter part of the catheter, the small outside diameter part of the catheter being adapted to be received in the small inside diameter part of the sheath.

26. A catheterization method, comprising:
providing a balloon catheter system, comprising
a balloon dilation catheter comprising a catheter shaft, and a balloon at a distal end of the catheter shaft,
a guidewire disposed in and having a limited range of longitudinal movement relative to the inner tubular element, and
a sheath adapted to be disposed around at least part of the catheter;
passing the balloon, guidewire, and sheath together through the patient's vasculature until the balloon is at a first location in a blood vessel;
at the first location forward of a distal end of the sheath, expanding the balloon;
advancing the sheath over the balloon at the first location;
after advancing the sheath over the balloon, withdrawing the catheter from the sheath while leaving the sheath in place.

27. The catheterization method as set forth in claim 26, after withdrawing the catheter, advancing a second guidewire through the sheath.

28. The catheterization method as set forth in claim 27, after advancing the second guidewire, withdrawing the sheath and leaving the second guidewire in place.

29. The catheterization method as set forth in claim 28, comprising advancing a second balloon catheter delivery system over the second guidewire.

30. The catheterization method as set forth in claim 26, after withdrawing the catheter, advancing a second balloon catheter delivery system through the sheath.

31. The catheterization method as set forth in claim 26, wherein the balloon catheter system is an advanceable, non-removable guide wire balloon catheter delivery system for a stent, and comprises an expandable stent mounted on the balloon, the method comprising expanding the stent by inflating the balloon at a second location.

32. The catheterization method as set forth in claim 31, wherein the stent is expanded without predilating the blood vessel.

33. The catheterization method as set forth in claim 31, wherein at least part of a distal part of the inner tubular element has a reduced outside diameter relative to a proximal part of the inner tubular element, the stent being mounted on the balloon over the reduced outside diameter part of the inner tubular element.

34. The catheterization method as set forth in claim 33, wherein at least part of a distal part of the guidewire in the distal part of the inner tubular element has a reduced outside diameter relative to a proximal part of the guidewire.
